SEQUENCE LISTING (10 sequences)

SEQ ID NO: 1 - DNA, 20 bp, Artificial Sequence
  primer_bind (1)..(20): PCR primer for universal detection of bacteria
agastttgatymtggctcag

SEQ ID NO: 2 - DNA, 20 bp, Artificial Sequence
  primer_bind (1)..(20): PCR primer for universal detection of bacteria
ccgtcaattcmtttgagttt

SEQ ID NO: 3 - DNA, 20 bp, Artificial Sequence
  primer_bind (1)..(20): PCR primer for universal detection of yeast
agaccgatagcgaacaagta

SEQ ID NO: 4 - DNA, 22 bp, Artificial Sequence
  primer_bind (1)..(22): PCR primer for universal detection of yeast
cttggtccgtgtttcaagacgg

SEQ ID NO: 5 - DNA, 22 bp, Artificial Sequence
  primer_bind (1)..(22): PCR primer for detecing S.epidermidis
cgtaaacgatgagtgctaagtg

SEQ ID NO: 6 - DNA, 20 bp, Artificial Sequence
  primer_bind (1)..(20): PCR primer for detecing S.epidermidis
aaggggcatgatgatttgac

SEQ ID NO: 7 - DNA, 19 bp, Artificial Sequence
  primer_bind (1)..(19): PCR primer for detecing B.subtilis
aagtcgagcggacagatgg

SEQ ID NO: 8 - DNA, 22 bp, Artificial Sequence
  primer_bind (1)..(22): PCR primer for detecing B.subtilis
ccagtttccaatgaccctcccc

SEQ ID NO: 9 - DNA, 22 bp, Artificial Sequence
  primer_bind (1)..(22): PCR primer for detecing E.coli
acctttgctcattgacgttacc

SEQ ID NO: 10 - DNA, 22 bp, Artificial Sequence
  primer_bind (1)..(22): PCR primer for detecing E.coli
caagaccaggtaaggttcttcg

(19) United States
(12) Patent Application Publication
(10) Pub. No.: US 2011/0065110 A1
(43) Pub. Date: Mar. 17, 2011

(54) Method For The Extraction And Purification Of Nucleic Acids On A Membrane

(75) Inventors: Roxana Isac, Strasbourg (FR); Frederic Marc, Itterswiller (FR)

(73) Assignee: MILLIPORE CORPORATION, Billerica, MA (US)

(21) Appl. No.: 12/849,455

(22) Filed: Aug. 3, 2010

Related U.S. Application Data
(62) Division of application No. 12/321,297, filed on Jan. 20, 2009.

(30) Foreign Application Priority Data
Jan. 21, 2008 (FR) 0850360

(51) Int. Cl. C12Q 1/68

(52) U.S. Cl. 435/6

(57) ABSTRACT

The invention relates to a method for the extraction and detection of nucleic acids on a membrane, making it possible to identify the microorganisms present in low concentration in a liquid or gaseous medium, as well as a novel lysis composition comprising guanidium chloride and between 0.1 and 1% N-Lauroyl-Sarcosine (NLS) making it possible to implement this method.

This application is a divisional of U.S. patent application Ser. No. 12/321,297 filed Jan. 20, 2009, the disclosure of which is incorporated herein by reference, and claims priority of French Patent Application No. 0850360 filed Jan. 21, 2008.

The present invention relates to a method for the extraction of nucleic acids which can be applied to a very small number of microorganisms filtered on a membrane, using a lysis composition comprising the combination of a guanidium salt and N-Lauroyl-sarcosine (NLS).

This method makes it possible to collect the nucleic acids of said microorganisms in a form suitable for detection in a specific manner by hybridization or amplification.

In the field of industrial or medical activities today, microbiological quality control of water and air, as well as tools and materials used or rejected, is required to comply with increasingly strict standards.

Accordingly, the industrial players and health authorities need to have tools at their disposal capable of detecting microbiological contamination as quickly as possible, so that remedial measures can be taken expeditiously and at a reduced cost.

Conventionally, microbiological monitoring is carried out on a agar culture medium. This type of culture, which is simple to implement, allows a germ enumeration to be carried out with the naked eye. It also makes it possible for the living microorganisms to be preserved, and if necessary for characterization terms to be carried out with the aim of defining the genus or the species of the different germs present.

These characterization tests generally consist of extracting the nucleic acids contained in the microorganisms and amplifying said nucleic acids in a specific manner, by one of a number of chain reaction techniques (PCR: polymerization chain reaction or LCR: ligature chain reaction) known to a person skilled in the art.

Nevertheless, although characterization tests by amplification of nucleic acids have a high level of reliability, the steps consisting of culturing the microorganisms, extracting their nucleic acids, and implementing the PCR technique, take a long time to perform.

In order to be visible to the naked eye, the microorganisms must be cultured for at least 24 hours, and sometimes longer, for slower-growing microorganisms such as methylobacterium sp. or because the germs have been stressed by the environmental conditions.

Moreover, the extraction of nucleic acids cannot be carried out directly on the gelose, which means that the microorganisms must be removed before their nucleic acids are extracted. In this case, the detection becomes qualitative and is no longer quantitative.

The recent development of lab-on-a-chip devices, based on hybridization technologies with nucleic acid probes fixed on a solid support, may allow a gain in time for the microorganism characterization step. However, these techniques still have a high cost price and do not give complete freedom from the nucleic acid extraction and transfer steps.

Under these conditions, over 24 hours must still be allowed for counting and characterization of the microorganisms, which is far too long in an industrial context where continuous monitoring of product quality is required.

In order to accelerate the detection of microorganisms present in liquid or gaseous media, or also on surfaces, the Applicant (Millipore Corporation) has for several years been developing a universal method for the detection of microorganisms, sold under the name of Milliflex® Rapid.

This method is described in Application WO 92-00145. It consists in that the microorganisms contained in a solution or in the air are retained at the surface of a membrane by passing the liquid or the air through the latter. The microorganisms are cultured at the surface of the membrane in contact with a agar culture medium for the time necessary to form a microcolony which is invisible to the naked eye. The cells forming the microcolony are then lysed in order to release their adenosine triphosphate (ATP) content. The ATP released is used as a marker in order to identify and quantify the living cells by ATP-bioluminescence. The ATP serves as a substrate to an enzyme producing a chemiluminescence reaction by which a light signal is obtained, then detected using an appropriate video interface (eg: LCD camera). The signal obtained, formed into an image, makes it possible to visualize in-situ the place on the membrane where the germ is developing, in a manner analogous with a standard counting method carried out on a Petri dish in a agar medium. This interface also makes it possible to quantify the number of microorganisms initially present in the liquid or gaseous sample analysed.

Detection of microorganisms in the Milliflex® system is called “universal” because it uses ATP (adenosine triphosphate), which is a substrate contained in all living cells. This technology makes it possible to detect the presence of contaminants quickly, whatever the microorganisms concerned, even at very low concentrations, for example on an industrial production line or in a clean room, so that the necessary decontamination measures can be taken within a short timescale.

However, using the Milliflex® system, does not currently make it possible to acquire, in parallel, reliable information on the identity of the microorganisms detected.

But this identification would be useful to determine the origin of the contamination in more detail and to define solutions to it.

The main obstacle to this identification resides in the fact that the detection by ATP-bioluminescence implemented in the Milliflex®, system is destructive of the microorganisms involved.

Indeed, during the reaction for detection by bioluminescence, the microorganisms are treated firstly, using an ethanol-based solution in order to extract the ATP molecules, then secondly by bioluminescence reagents. Moreover, the membrane is dried between these two steps in order to carry out the bioluminescence reaction. The result of these treatments is that the microorganisms are killed, which prevents their subsequent culture for carrying out standard characterization tests (antibiotic resistance, metabolic markers, gram reactions, etc.).

The inventors have therefore undertaken, according to the present invention, to characterize the microorganisms detected by retrieving the nucleic acids of the microorganisms treated by bioluminescence in order to proceed to their specific detection by hybridization or by amplification, in particular by PCR.

However, the techniques of amplification or hybridization of the nucleic acids require purified samples of DNA or RNA which are sufficiently concentrated to make it possible to proceed reliably to the desired detection reactions.

In the particular case of detection on a filtration membrane, the difficulties in retrieving these nucleic acids are numerous, in particular due to the fact that:
the microorganisms are dispersed at the surface of the membrane in the form of one or more micro-colonies, each one of which comprises a small number of microorganisms, generally less than 10³ cells; and that
the cells containing the nucleic acids to be extracted are dried, and moreover have been previously treated with agents and salts capable of inhibiting or reducing the yield of the hybridization or the PCR reactions.

These constraints mean that the nucleic acids must be recovered in solution on the membrane, purified and concentrated.

Many techniques exist in the prior art for extracting, purifying and concentrating the nucleic acids contained in cells, which differ according to the nature, RNA or DNA, of the nucleic acids.

The large majority of these techniques require an initial treatment by a lysis buffer, then a step of precipitation of the nucleic acids in solution. The purpose of the lysis buffer is to rupture the cell membranes and solubilize the proteins, while the precipitation makes it possible to separate the nucleic acids from the other cell constituents in solution.

The most usual method for extracting the RNA (more fragile than DNA) is for example, the one known as the “phenol-chloroform” method.

According to this technique, the cells are firstly placed in contact with a lysis buffer comprising a detergent-proteinase K mixture, the purpose of which is to dissociate the cell constituents and release the nucleic acids.

The lysis buffer used generally comprises as a detergent, dodecyl sodium sulphate (SDS), which is the cell lysis agent, as well as an activator of proteinase K such as sarkosyl, Tween® 20, or Nonidet P40.

The nucleic acids contained in the cell lysate obtained are then separated from the proteins, by applying a mixture of phenol, a powerful deproteinizing agent, and chloroform, which is an organic solvent. After centrifugation, the nucleic acids are solubilized in the organic phase, while the proteins are collected at the interphase between the aqueous phase and the organic phase.

After transfer of the aqueous phase into another tube, the nucleic acids are treated with an chloroform-isoamylic alcohol mixture. The effect of this is to eliminate the traces of phenol, an organic compound which has the drawback of being not only a toxic product but also an inhibitor of certain enzymes, including polymerase.

The nucleic acids are then precipitated by the addition of a precipitation agent, typically absolute ethanol at −20° C., at the rate of 2.5 times the volume of the lysis buffer, or the isopropanol, at the rate of 0.7 volume.

A subsequent washing of the precipitated nucleic acids in 70% ethanol is indispensable for removing the salts.

The precipitate is then taken up in a buffer with a low ionic strength, in general, a Tris-EDTA buffer (10/1 mM).

Although the Phenol/chloroform method is the reference method in respect of nucleic acid extraction, the latter has the drawback of comprising a number of steps and using phenol and chloroform, which are both volatile and toxic products which must be handled with care. When seeking to extract genomic DNA, which is less fragile than RNA molecules, the trend is therefore to use a lysis buffer typically comprising between 4 and 6 M guanidium thiocyanate, 10 mM EDTA, between 2 and 6% NLS and 50 mM Tris-HCl (neutral pH) (so-called modified Crude Susan method) [Chirgwin, J. et al. (1979) Isolation of biologically active ribonucleic acid from sources enriched in ribonuclease, Biochem. 18:5294-5299]. A lysis buffer of this type makes it possible to carry out precipitation of the nucleic acids without using phenol-chloroform, directly in the cell lysate [Jeanpierre, M. (1987) A rapid method for the purification of DNA from blood, Nucleic Acids Research 15(22):9611].

Nevertheless, the quality of the nucleic acids obtained under these conditions, and the yield of the reactions are not constant, in particular on account of the larger volume of the aqueous phase and the cell constituents load, which is variable according to the nature of the microorganisms. It can be then necessary to carry out a second precipitation of the nucleic acids in order to obtain a good-quality DNA [De Nedjma, A. (2005) Principe de Biologie Moleculaire in Biologie Clinique, Elsevier & Masson].

Certain techniques use micro-columns containing silica beads, or a similar solid adsorption phase, in order to facilitate the steps of washing and recovery of the nucleic acids. However, the principle is the same, since a step of precipitation of the nucleic acids on the adsorption phase is also required.

The above mentioned techniques are not suitable for the preparation and detection of small quantities of nucleic acids. In fact, as stated above, when the nucleic acids are too diluted, their precipitation becomes more random and there is an increased risk of the nucleic acids being lost during the purification method.

The purpose of the present invention is therefore to achieve a simple, rapid and effective method for preparing nucleic acids, both DNA and RNA, from a small number of microorganisms, in order to then carry out their characterization.

Thus the present Application discloses a method for the extraction and purification of genomic DNA which can be applied, for example, to cells filtered on a membrane, by which the cells are treated in solution using guanidium chloride and a limited quantity of NLS. According to this method, the nucleic acids are purified on a membrane, without continuing to a precipitation step, and the DNA retained by the membrane is recovered in a volume of pure or buffered water in a form suitable for detection by hybridization or by PCR.

Surprisingly, the best detection results were obtained by the inventors using a low concentration of NLS in the composition, much lower than that usually found in lysis compositions. It has been noted in particular that at this low concentration, the NLS made it possible to avoid the problems associated with clogging the membrane during the purification step, and also to facilitate the retrieval of nucleic acids at the end of the method.

The invention was implemented more particularly with the aim of characterizing, by PCR, a very small number of microorganisms filtered on a membrane, up to the detection threshold of 1 CFU of the Milliflex® Rapid system. However, the invention can be applied outside the particular context of membrane microbiology, whatever the type of cells involved, for preparing nucleic acids, in particular with the aim of carrying out hybridization or amplification reactions. The nucleic acid preparations obtained can be used, for example, to carry out PCR reactions or isothermal amplification reactions of the LAMP type, or also reactions involving RNA molecules, such as amplification of the NASBA or TMA type, it being understood that the RNA sequences can be transcribed to DNA by a retro-transcription step.

The following figures illustrate the results of the implementation examples of the invention described in the present Application.

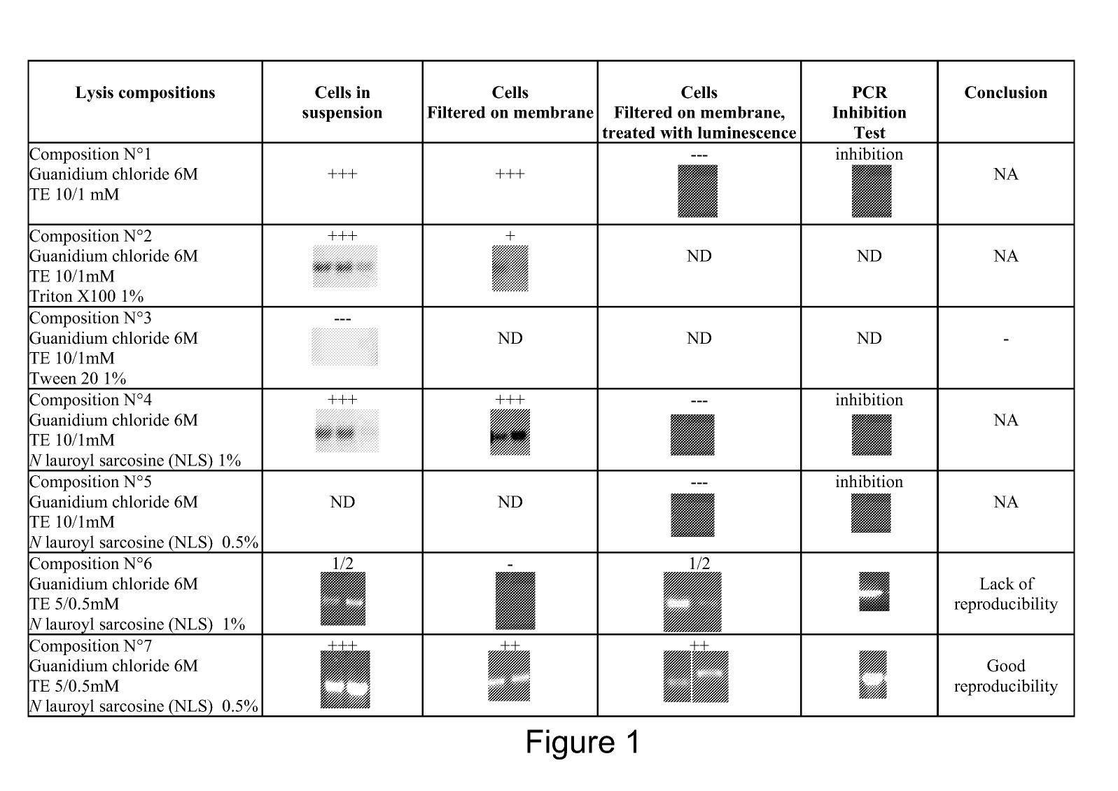

FIG. 1: Comparison of the effectiveness of lysis compositions 1 to 7 used for the detection of microorganisms by PCR. In all cases, the DNA has been purified on a cellulose membrane contained in a (Microcon®) centrifuge tube following treatment of the cells by the corresponding lysis composition. The experiment was carried out on fresh cells sampled on a culture (cells in suspension), fresh cells sampled on a PVDF filtration membrane or on cells sampled on a PVDF membrane after treatment with ATP-bioluminescence (according to the Milliflex® Rapid method). For the PCR-inhibition control, pure DNA was introduced into the lysis compositions, then purified on a membrane, as in the case of the other preparations. The compositions were tested at least twice per experiment. The cells detected are E. coli bacteria. The PCR results are given in the form of photos showing the reproducibility and intensity of the amplification. +: positive amplification; −: negative amplification, ½: absence of reproducibility; ND: not performed; NA: negative result on membrane.

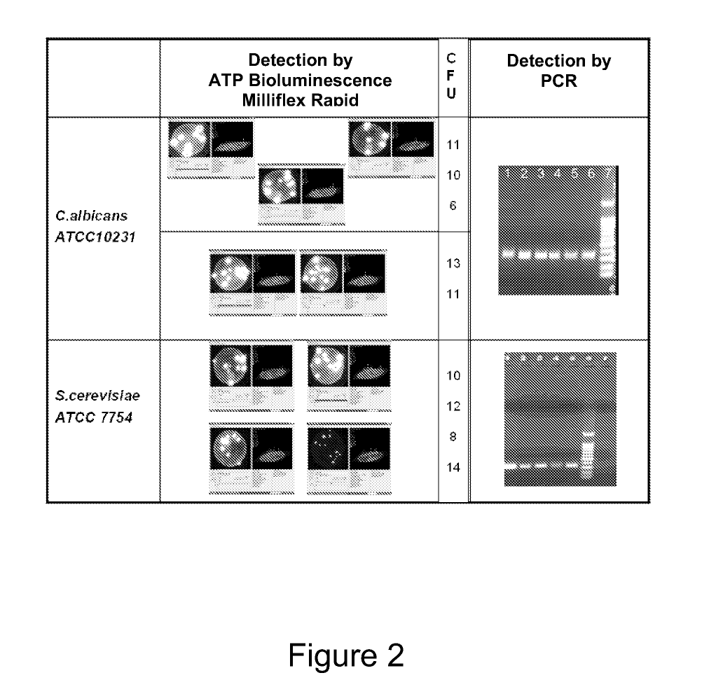

FIG. 2: Test results of the nucleic acid extraction and detection method according to the invention, as it relates to C. albicans and S. cerevisiae yeasts. The agarose gels show a good reproducibility of the amplifications by PCR. The wells are numbered from left to right: 1: positive PCR control (pure DNA of C. albicans), 2 to 6 (top gel) or 2 to 5 (bottom gel): different samples tested; 7 (first gel) or 6 (second gel): 1000 by plus ladder. The PCR reactions are carried out with universal primers, making it possible to detect unicellular fungi. In the centre the synthesis images of the membrane are shown resulting from the universal detection by ATP-bioluminescence carried out for each PCR-tested sample.

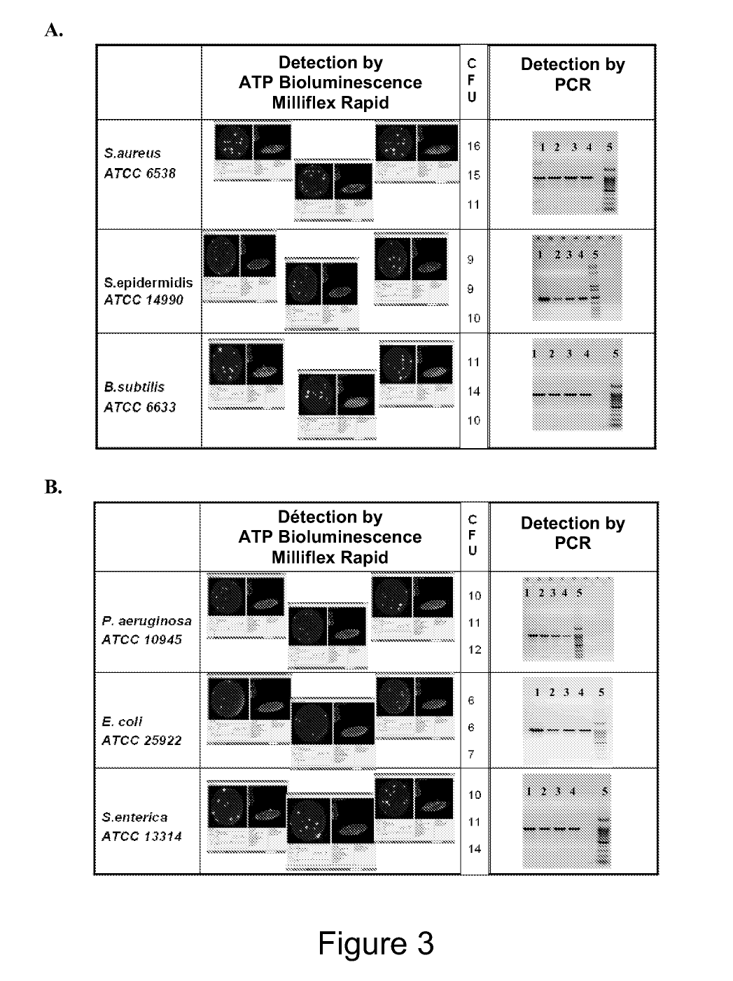

FIG. 3: Test results of the nucleic acid extraction and detection method according to the invention, as it relates to the genomic DNA (A) of the gram-positive bacteria S. aureus, S. epidermidis, B. subtilis (B) the gram-negative bacteria P. aeruginosa, E. coli, S. enterica. The wells of the agarose gels are numbered from left to right. 1: positive PCR control; 2 to 4: PCR carried out for 3 independent samples with primers common to gram-positive or gram-negative bacteria; 5: 100 pb plus ladder. In the centre the synthesis images of the membrane are shown resulting from the universal detection by ATP-bioluminescence carried out for each PCR-tested sample.

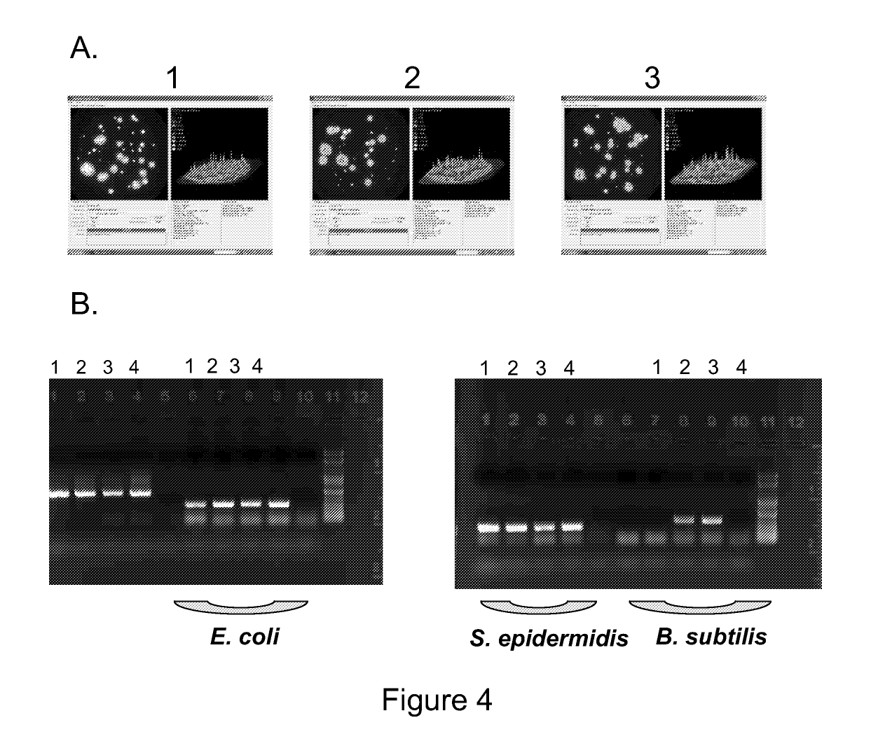

FIG. 4: Test results of the nucleic acid extraction and detection method according to the invention, as it relates to the genomic DNA of populations of cells comprising a mixture of E. coli and S. epidermis. A: enumeration of the mixture of the cells in three populations by ATP-bioluminescence following the Milliflex® method. B: detection by PCR amplification according to the invention, visualized on agarose gel. 1: PCR positive control; 2 to 4: PCR carried out for each of the three populations; 5: negative control; 6: 100 pb plus ladder. 1^{st} series: PCR carried out using universal primers on E. coli and S. epidermis; 2^{nd} series: PCR carried out with specific primers on E. coli; 3^{rd} series: PCR carried out with specific primers on S. epidermis; 4^{th} series: PCR carried out with specific primers on B. subtilis.

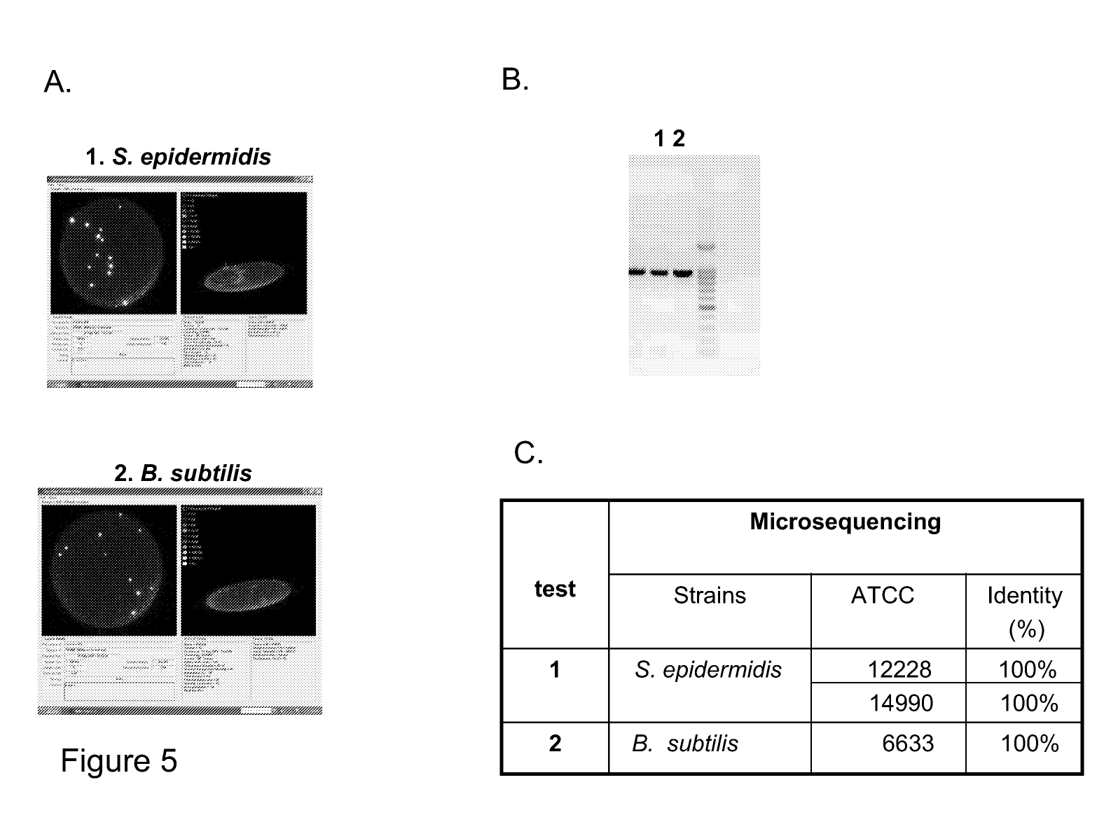

FIG. 5: Detection by ATP-bioluminescence (Milliflex Rapid®) then by PCR and micro-sequencing according to the invention. A: count of 13 CFU of S. epidermidis and 9 CFU of B. subtilis. B: visualization on gel of the products of PCR amplification carried out on the extracts of genomic DNA purified from the same cells of S. epidermis and B. subtilis as those detected on the membrane in A. C: Results of the micro-sequencing carried out on the amplification products visualized in B.

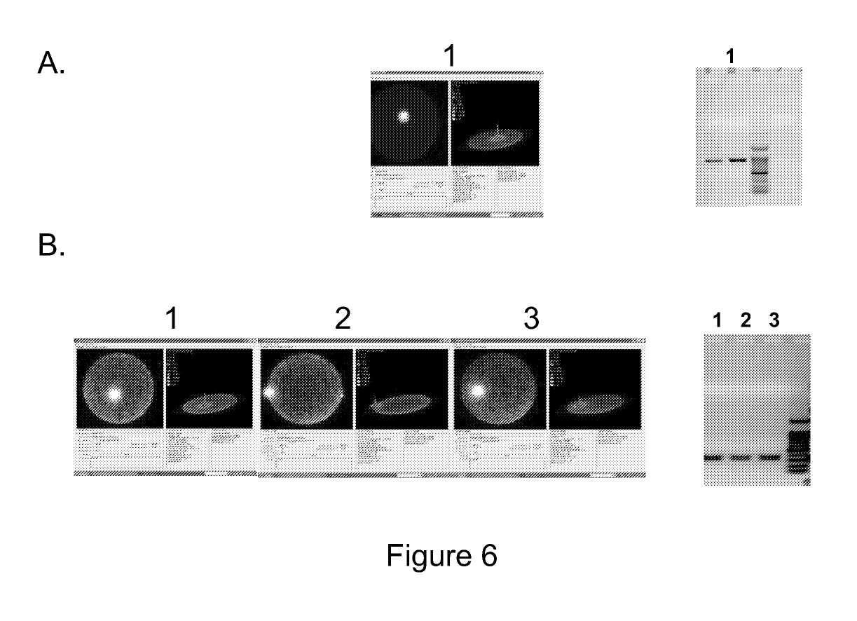

FIG. 6: Results of the sensitivity tests relating to PCR detection of the unicellular fungi and bacteria according to the invention. The genomic DNA corresponding to 1 CFU, detected firstly by bioluminescence, is extracted then detected by PCR according to the method of the invention. A: Positive result of PCR obtained for a bacterial sample (E. coli) using universal primers specific to the bacteria. B: Positive results of PCR obtained for 3 different samples of unicellular fungi (S. cerevisiae)

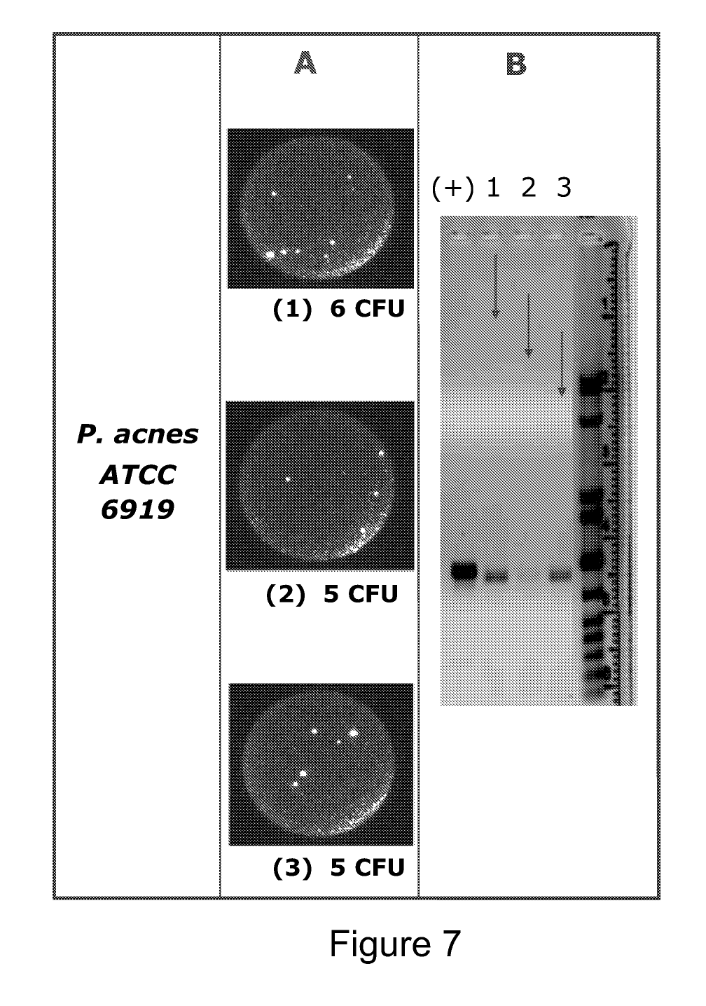

FIG. 7: Detection by ATP bioluminescence and PCR of the gram-positive bacteria P. acnes. A: Result of the detection by ATP bioluminescence in three samples making it possible to assess the number of bacteria detected at 5 and 6 CFU. B: Positive results of PCR obtained using the nucleic acid preparations extracted respectively from these three samples by the extraction method according to the invention.

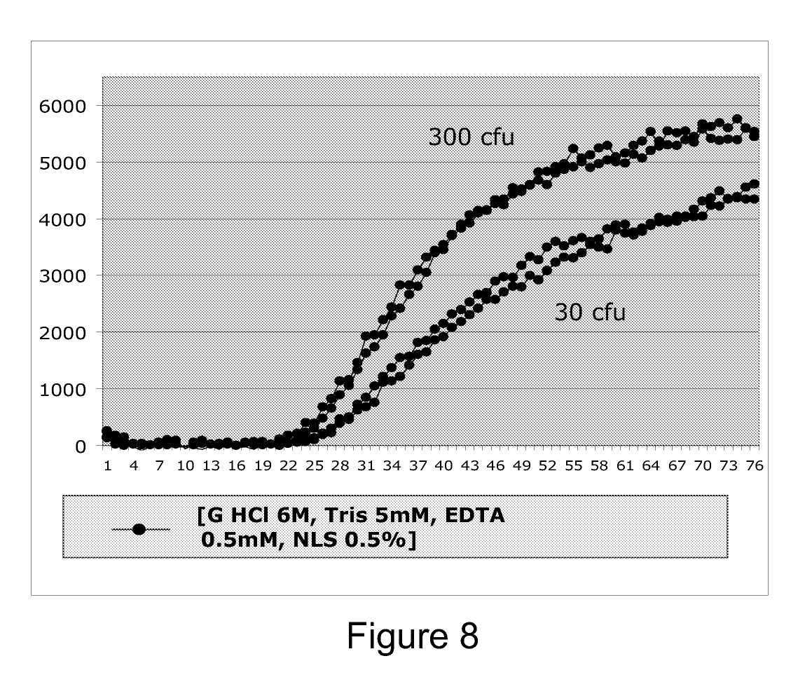

FIG. 8: Real-time accumulation profile of the RNA amplification fragments resulting from the amplification by TMA of RNA contained in two nucleic acid preparations extracted from Pseudomonas aeruginosa according to the invention. The preparations correspond respectively to a number of bacteria of approximately 30 and 300 CFU.

The subject of present invention is thus a method for the extraction and detection of nucleic acids on membrane, making it possible to identify the microorganisms present in a low concentration in a liquid or gaseous medium, or even present on a surface.

The method according to the invention relates more particularly to the detection of microorganisms transferred onto a membrane by filtration of a liquid or gaseous sample through the latter, or by placing said membrane in contact with a medium or a surface capable of containing or comprising microorganisms. It aims more particularly at solving the problem of identification of germs dispersed at the surface of the membrane.

The method according to the invention is particularly appropriate for the identification of microorganisms previously detected on a membrane, for example using a non-specific method such as ATP bioluminescence used in the Milliflex rapid method.

The method according to the invention consists of extracting and purifying the nucleic acids of a small number of cells, living or dead. It can be applied, outside the particular context of the detection of microorganisms on a membrane, to any cell from which it is sought to extract nucleic acids in a rapid and effective manner. This method is particularly useful in order to obtain RNA and/or DNA nucleic acids, in a form suitable for producing the hybridization or amplification reactions necessary for their characterization.

This method comprises the following steps, during which:
a) cells are treated with guanidium chloride and N-Lauroyl-sarcosine (NLS) in solution, in order to lyse the cells and release the nucleic acids that they contain;
b) the nucleic acids are separated from the cell lysate obtained by the filtration of said lysate through a membrane;
c) the nucleic acids retained by said membrane are recovered in solution in water or a weakly ionized aqueous solution.

A “cell” is defined herein as a biological entity of small size comprising a cytoplasm delimited by a membrane and containing genetic material in the form of nucleic acids.

By “microorganism” is meant within the meaning of the present invention, a cell having a microscopic size, i.e. a size comprised between 0.5 and 5 microns, having a metabolic and reproductive potential, such as algae, unicellular fungi, protozoans, mycetes, bacteria or also gametes.

The microorganisms sought are more particularly pathogenic bacteria gram-positive or gram-negative bacteria of the genera Pseudomonas, Escherichia, Legionella, Salmonella, Listeria, Bacillus, Streptococcus, Vibrio, Yersinia, Staphylococcus, Mycobacterium, Shigella, Clostridium, Campylobacter, or Aeromonas; protozoans of the genera Giardia, Entamoeba, Cryptosporidium, Cyclospora; mycoplasms of the genera Mycoplasma and Ureaplasma, fungi of the genera Saccharomyces, Aspergillus, Candida or Penicillium.

The treatment of the cells in step a) by guanidium chloride and N-Lauroyl-sarcosine can be carried out, by one and then the other of these compounds or even simultaneously. According to a preferred aspect of the invention, the treatment is carried out using a lysis composition comprising an NLS concentration comprised between 0.1 and 1% of the total weight of the composition.

The guanidium chloride is a chaotropic agent which is used as a lysis agent. It has the double effect of denaturing the proteins, in particular the membrane proteins, and producing osmotic shock. It is generally used at a concentration comprised between 3 and 8 moles per litre, more preferably between 4 and 6 moles per litre of lysis composition.

N-Lauroyl-sarcosine (Sarkosyl NL-97 or NLS) is a sodium salt of N-methyl-N-(1-oxododecyl)glycine (C₁₅H₂₈NO₃Na). This molecule is a detergent often used in lysis buffers in combination with proteinase K, at concentrations of the order of several moles per litre, for solubilizing the proteins, in particular the membrane proteins.

According to the present invention, NLS is not used as a lysis agent or at least not principally. In fact, according to the invention, NLS is preferably used at a concentration comprised between 0.1 and 1% of the final weight of the lysis composition, more preferably between 0.1 and 0.5%, i.e. at a concentration where the effect on cell lysis is limited.

As shown in the examples of the present patent application (see in particular the results in FIG. 1), the addition of NLS at this concentration makes it possible to improve the extraction and purification yield, in particular in steps b) and c) of the above method, and consequently the characterization of microorganisms which can be carried out subsequently using the nucleic acid preparation obtained.

NLS is an anionic detergent which denatures almost all the non-covalent interactions in the proteins and tends to solubilize the proteins. Without being bound by theory, the inventors think that NLS facilitates the separation of the DNA and the proteins during filtration of the cell lysate through the membrane, and the rinsing of the DNA which follows.

By “cell lysate”, is meant here the lysed cells in solution with the lysis composition, on completion of the treatment in step a).

Preferably, the lysis composition according to the invention also comprises between 0.1 and 1 mmol·l⁻¹ of ethylenediaminetetraacetic acid (EDTA). Although EDTA is recognized as a substance capable of inhibiting the activity of numerous enzymes, in particular the polymerase in PCR reactions, it has been discovered in experiments carried out by the inventors that the presence of EDTA makes it possible to improve the lysis of not only gram-negative, but also gram-positive microorganisms. In principle, EDTA makes it possible to destabilize the membrane of bacteria by sequestering the divalent ions.

In the present application, “membrane” denotes a synthetic support having two surfaces, the pores of which have a known average diameter. Such a membrane has a high surface/volume ratio. The thickness of the membrane is generally constant, between 90 and 200 microns.

Such a membrane can be single- or multi-layer. It is generally constituted of one or more materials chosen from polytetrafluoroethylene, polyvinylidene fluoride (PVDF), polycarbonate, polyamide, polyester, polyethersulphone, acetylcellulose and nitrocellulose.

The membrane designated in step b) of the DNA extraction method according to the invention is generally a membrane made of cellulose called an ultrafiltration membrane i.e. a membrane which has a molecular weight nominal limit between 3′000 and 100,000 daltons, preferably between 50,000 and 100,000 daltons. An example of this type of membrane is the one with which Microcon® centrifuge tubes (Millipore Corporation) are provided.

According to the invention, separation of the nucleic acids from the cell lysate is carried out through the membrane, under the effect of a negative or positive pressure. By way of example, passage of the lysate can be carried out by means of a low pressure on one of the surfaces of the membrane, using a vacuum pump. This can also activated under the effect of centrifugation, i.e. by applying a centrifugal force to the cell lysate at the surface of the membrane. The centrifugation effect is generally obtained by placing the membrane on a support such as a centrifuge tube, placed in a centrifuge.

The invention moreover provides that between steps b) and c) of the method, a step of washing the nucleic acids retained by the membrane can be carried out, using a washing solution such as water or a weakly ionized aqueous solution. The washing solution is removed after passing through the membrane.

In step c) the nucleic acids can be easily recovered by passing the water or the weakly ionized aqueous solution through the membrane, in the opposite direction to that applied in step b) or even that of the additional washing step mentioned above. When step c) is facilitated by the application of a negative or positive pressure, or even by centrifugation, all that is required is to place the support on which the membrane is fixed in the opposite direction to its original one, and place a drop of the weakly ionized solution on the surface of the membrane.

The force exerted by the pressure or the centrifugation forces the water through the membrane pores and releases the nucleic acids from the membrane. The nucleic acids are then collected in the drop of water thrown to the bottom of the centrifuge tube.

In this case, by weakly ionized is designated a solution the concentration in salts of which per litre is less than 10⁻² moles/L, more preferably less than 5·10⁻³ moles/L.

More particularly, the invention relates to a method for the detection of one or more cells in a liquid or gaseous medium, characterized in that it comprises the following steps:

a) a sample of the liquid or gaseous medium is filtered through a membrane in order to retain the cells contained in said sample on said membrane;

b) the cells retained in step a) are treated with guanidium chloride and N-Lauroyl-sarcosine (NLS) in solution, in order to obtain a cell lysate containing the nucleic acids released from said cells;

c) the nucleic acids of the cell lysate obtained in step b) are separated by filtration of said lysate through said membrane, or through another membrane;

d) the nucleic acids retained by the membrane in step c) are recovered in water or in an weakly ionized aqueous solution;

e) the nucleic acids recovered in step d) are detected.

Steps b) to d) of said detection method preferably correspond to the method for the extraction and purification of nucleic acids according to the invention, i.e. those carried out according to steps a) to c) of the method described above.

The purpose of the detection method is to count the cells, more particularly the microorganisms present in a liquid medium such as water, or gaseous medium such as air, while determining their identity as far as possible, (family, genus, species, etc.).

Liquid or gaseous medium denotes any fluid capable of being filtered by applying a pressure difference across a membrane having pores of a an average diameter generally comprised between 0.1 and 1.5 microns, preferably between 0.15 and 0.8 microns, more preferably between 0.2 and 0.6 microns. Such a fluid can consist of pure solutions forming part of the manufacture of sterile products but also complex solutions (potable water, serum, urines, amniotic fluid, etc.) or also gaseous mixtures such as atmospheric air.

The present method can be applied to the field of diagnostics for the analysis of samples originating from animals or patients.

In step a) of the detection method, cell transfer is generally carried out on a so-called filtration membrane, i.e. a membrane which is permeable to liquid or gaseous media, the average pore size of which, however, allows the retention of at least 80%, preferably 90% of the cells or microorganisms sought. Preferably, this membrane is mainly constituted of PVDF (polyvinylidene fluoride), cellulose or polyethylenesulphone. More preferably, it is a filter membrane made of PVDF, of the type sold by the firm Millipore under the trade name Milliflex and the references MXHVWP124 or RMHVMFX24.

According to a preferred aspect of the invention, the membrane used for separating the nucleic acids of the lysis composition in step c) is different from that used in step a). The membrane used in step a) can moreover be placed in contact with a nutrient medium so that the cells or microorganisms retained by said membrane can grow on its surface between steps a) and b). The nutrient medium is then generally a gelose medium, the nutrients of which reach the cells by capillary action. While multiplying on the surface of the membrane, formation of the cells into micro-colonies takes place.

As is also provided for in the Milliflex technology, a detection step, making it possible to count the microorganisms on the membrane, can be previously implemented before cell lysis in step b), for example, by extracting the ATP from the cells between step a) and step b), and by reacting this ATP with a bioluminescent reagent comprising luciferine and luciferase.

According to the invention, the nucleic acids extracted are constituted by RNA and DNA, in particular messenger and ribosomal RNA and genomic DNA. The method according to the invention has the advantage of being able to use RNA as well as DNA extracted from the cells by the method according to the invention.

Detection of the nucleic acids in step e) is preferably carried out by a specific hybridization of all or part of the nucleic acids recovered in step d) using a primer or a probe capable of being specifically marked.

According to the invention, by probe is meant macromolecules capable of recognizing and combining with one of the categories of nucleic acids mentioned above, making their marking possible.

According to the invention, by primer is meant macromolecules capable of recognizing and combining with one of the categories of nucleic acids mentioned above, in order to initiate amplification reactions of said nucleic acids.

The probe or the primer used is generally a macromolecule capable of hybridizing with the DNA or RNA sequences present among the extracted nucleic acids with a certain degree of specificity. The invention envisages any type of probe or primers known to a person skilled in the art which can give rise to a specific hybridization with nucleic acids such as for example simple oligonucleotides, oligonucleotides of the 2′O-methyl-RNA type, probes or primers of the PNA type (probes constituted by a polypeptide chain substituted by purine and pyrimidine bases) [Nielsen P. E. et al. Science (1991) 254:1497-1500] or LNA (oligonucleotides comprising one or more monomers of 2′-O-4′-C-methylene-β-D-ribofuranosyl) such as described in patent EP 1 013 661.

By amplification reaction is designated the enzymatic reactions making it possible to synthesize from said nucleic acids, macromolecules the presence of which can be detected by standard methods, for example by chromatography on agarose gel or acrylamide, micro-sequencing or fluorescence.

The method according to the invention can therefore comprise in step e) a step of amplification of all or part of the nucleic acids recovered in step d) by one of the techniques known to a person skilled in the art such as PCR (Polymerization Chain Reaction) or also an amplification reaction carried out isothermically according to the techniques well known to a person skilled in the art such as, for example, amplifications of the NASB type [Compton, J., Nature (1991) 350:91-92], TMA or the LAMP type [Notomi T., Nucleic Acids Research (2000), 28(12): e63].

Such an amplification can prove useful for the optimum detection of microorganisms by increasing the quantity of the nucleic acids available for the purposes of detection. If this step of amplification is highly specific, it moreover makes it possible to identify the microorganisms with greater selectivity.

The nucleic acid preparations obtained according to the method of the invention are particularly suitable for the implementation of a detection of nucleic acids by amplification.

A subject of the invention is also a kit for the extraction of nucleic acids, characterized in that it comprises:
a membrane, preferably made of cellulose or PVDF.
a lysis composition as defined previously.

Advantageously, this kit moreover comprises one or more specific probes or primers for carrying out the detection of the nucleic acid extracted, in particular by hybridization or by PCR. Optionally, this kit can also comprise a second membrane for the filtration of a liquid or gaseous medium. This kit makes it possible for the method according to the invention to be implemented.

Such a kit is particularly useful for implementing the methods for extraction and purification of DNA, and subsequently, for identifying the cells or microorganisms under the conditions defined previously.

The following examples are intended to complement the description of the present invention without imposing any limitation thereto.

### EXAMPLES

### Materials and Methods

### Microorganisms Tested

The microorganisms used in the detection test described hereafter were obtained from the American microorganism collection ATCC. The reference number of the strains is given in Table 1.

TABLE 1
Microorganisms used

| Microorganisms | Gram | ATCC |
|---|---|---|
| Escherichia coli | − | 8739 |
| | | 25922 |
| Pseudomonas aeruginosa | − | 9027 |
| Salmonella enterica | − | 13314 |
| Staphylococcus aureus | + | 6538 |
| Staphylococcus epidermidis | + | 14990 |
| Bacillus subtilis | + | 6633 |
| Propionibacterium acnes | + | 6919 |

These strains were kept at −80° C. in a suitable medium (Sigma, ref C6039). They were then cultured and diluted in a peptone-based medium (Biomerieux, ref. 42111) in order to obtain the required density.

### Total Flora Count

The microorganisms were counted according to two methods:

1—Method of Enumeration on Gelose Medium:

Petri dishes containing a gelose medium inoculated with 100 μl of a diluted culture are incubated for 24 to 48 hours at 35°+/−2.5° C. for bacteria, 48 to 72 h at 25°+/−2.5° C. for the yeasts and the moulds. The colonies are visually enumerated on three different dishes in order to determiner the concentration of the diluted cultures which were used for their inoculation

2—Method by Filtration:

The apparatus and consumables associated with the Milliflex® system sold by Millipore were used for filtering the same 100 μl of diluted culture on a membrane made of cellulose (MCE) (Millipore, ref. MXHAWG124) or of polyvinylidene fluoride (PVDF) (Millipore, ref. RMHVWP124), the average pore diameter of which is 0.45 μm. The following procedure is followed, under sterile conditions:
1. 50 mL of a sterile solution comprising 0.9% NaCl is placed in the funnel;
2. then the 100 μl of diluted culture is added;
3. a further 50 mL of a sterile solution is added, comprising 0.9% NaCl for homogenization;
4. filtration is carried out using a vacuum pump through the cellulose membrane and the membrane is transferred onto a gelose medium cassette;
5. the microorganisms are allowed to develop on the membrane, either for a few hours for counting by ATP bioluminescence (rapid microbiology) or for 1 or 2 days for counting with the naked eye.

The automated rapid detection system (Milliflex Rapid Microbiology Detection system) is then used for detecting the microcolonies which develop on the PVDF membrane, using the ATP bioluminescence technique. This technique uses a lysis reagent to release the ATP of the cells and a bioluminescence reagent comprising luciferase and luciferine using the ATP as a reaction substrate. The membranes are treated by spraying 35 μl of each of the reagents by using a suitable spray (Milliflex Rapid Autospray Station; Millipore, ref. MXRP SPRKT). The photons produced by the reaction between the ATP and the bioluminescence reagent are detected in a darkroom by a CCD camera connected to a software-managed interface, making it possible to reconstitute the membrane as a synthesis image (Milliflex Rapid software v3.0). The minimum ATP concentration measured is 200 attomoles, i.e. the equivalent of the quantity of ATP extracted from a yeast or from 100 bacteria.

### Extraction of the Nucleic Acids Contained at the Surface of the Milliflex Membrane

The cells present on the PVDF membrane after the bioluminescence detection step described above at point 1.2 are taken up in a lysis buffer. Several lysis buffers have been tested. These buffers have been formulated from one or more of the products sold by the company SIGMA, chosen from the following, at different concentrations: guanidium thiocyanate (Ref. 50980), guanidium hydrochloride (Ref. G9284), Tris EDTA buffer (Ref. 86377), Triton X100 (Ref. T8787), Tween 20 (Ref. P-7949) and N-lauroyl sarcosine (Ref. L 7414).

The lysis protocol is as follows:
1. The Milliflex membrane is placed on a cassette (ref Millipore cat number MPRBLB100) in such a way as to form a sealed unit, inside which 1 to 1.5 ml of the lysis composition is injected, using a syringe.
2. The membrane is maintained in contact with the lysis composition for 1 h at 55° C. under gentle agitation at 10-12 rpm.
3. The cell lysate is recovered using the syringe.

### Purification of the Nucleic Acids

Two methods were used: the standard method based on a precipitation of the nucleic acids with isopropyl alcohol (IPA, Sigma ref. 19030) then a treatment with ethanol (Prolabo, ref. 20 824.365) so-called “modified Crude Susan” method. According to this method that the inventors have modified, the cells are treated in the absence of proteinase K with a lysis composition comprising:

Guanidium thiocyanate 4M

NLS 2%

Tris, pH=7.6 50 mM

EDTA 10 mM

β-mercaptoethanol 1%

The isopropyl alcohol is added to the cell lysate thus treated, volume for volume. The mixture obtained is centrifuged for 30 min at 13200 rpm at 15° C. The supernatant is then removed from the centrifuge tube, the pellet formed by the precipitate is rinsed with 400 μl ethanol at 70%, then to centrifuged again for 10 min at 13200 rpm at 15° C. The supernatant is discarded and the pellet dried at ambient temperature for 20 minutes. Finally, the nucleic acid precipitate is dissolved in 50 μl of pure water.

The other method according to the invention consists of using an ultra-filtration membrane fitted in a centrifuge tube. This type of membrane is available under the trade name of Microcon® filter (Millipore, ref. 42413). The Microcon® filter contains a regenerated cellulose membrane, the pores of which allow the passage of molecules of nominal molecular weight less than 100,000 Da (MWCO). The consumable has a capacity of 500 μl.

The nucleic acid purification protocol is as follows:
1. 500 μl of cell lysate are placed on the membrane in the filter, which is subjected to centrifuging at 500×g for 25 to 30 minutes at ambient temperature;
2. the cell lysate which has passed through the membrane is discarded;
3. a volume of 450 μl of purified water (MilliQ) at 55° C. is placed on the membrane in the filter;
4. the filter is again subjected to centrifuging for 20 minutes at 500×g at ambient temperature;
5. the water which has passed through the membrane is discarded;
6. the same operation as in 4. and 5. is repeated;
7. the volume of water remaining on the surface of the membrane is adjusted to 50 μl;
8. the filter is vortexed for a few seconds;
9. the filter is turned over and placed in a new 1.5 ml centrifuge tube;
10. The filter is centrifuged for 3 minutes at 1,000×g;
11. the nucleic acids are recovered in the aqueous solution present in the 1.5 ml tube.

It is recommended that the membrane should not dry out during these operations.

### Amplification of the DNA by PCR

The PCR amplifications were carried out using the Qiagen PCR kit (Ref. 201223), following the manufacturer's instructions. The volumes of reagents used are shown in Table 2. The sequence of the primers manufactured by Eurogentec is shown in Table 3, as well as their hybridization temperature.

TABLE 2
PCR reagents used

| Mix components | Concentration | Volume |
|---|---|---|
| Taq buffer | 10x | 2.5 μl |
| dNTP | 10 mM | 1 μl |
| Primer forward (1/10) | 100 μM | 1 μl |
| Primer reverse (1/10) | 100 μM | 1 μl |
| Taq DNA polymerase | 5 U/μl | 0.2 μl |
| DNA Template | | 19.3 μl |
| QSF | | 25 μl |

TABLE 3
sequences of the primers and target of said primers.

| | | | | Hybridization |
|---|---|---|---|---|
| Primers | Sequences 5′ > 3′ | | Target DNA | T° C. |
| Bact-F | AGASTTTGATYMTGGCTCAG | (SEQ ID N^{o} 1) | 16S | 52° C. |
| Bact-R | CCGTCAATTCMTTTGAGTTT | (SEQ ID N^{o} 2) | ribosomalDNA | |
| YM-F | AGACCGATAGCGAACAAGTA | (SEQ ID N^{o} 3) | 16S | 47° C. |
| YM-R | CTTGGTCCGTGTTTCAAGACGG | (SEQ ID N^{o} 4) | ribosomalDNA | |
| S.epi-F | CGTAAACGATGAGTGCTAAGTG | (SEQ ID N^{o} 5) | S.epidermidis | 64° C. |
| S.epi-R | AAGGGGCATGATGATTTGAC | (SEQ ID N^{o} 6) | 16S rDNA | |
| B.sub-F | AAGTCGAGCGGACAGATGG | (SEQ ID N^{o} 7) | B. subtilis | 60° C. |
| B.sub-R | CCAGTTTCCAATGACCCTCCCC | (SEQ ID N^{o} 8) | 16S rDNA | |
| E.coli-F | ACCTTTGCTCATTGACGTTACC | (SEQ ID N^{o} 9) | E.coli | 62° C. |
| E.coli-R | CAAGACCAGGTAAGGTTCTTCG | (SEQ ID N^{o} 10) | 16S rDNA | |

The PCR conditions for Bact-F and Bact-R are as follows:

(Universal Detection for the Bacteria)
1 cycle at 95° C. for 05:00 minutes;
35 cycles

(94° C. for 00:30, 52° C. for 00:40, 72° C. for 01:10);
1 cycle at 72° C. for 20:00 minutes.

The PCR conditions for YM-F and YM-R are as follows:

(Universal Detection for the Yeasts)
1 cycle at 95° C. for 05:00 minutes;
35 cycles

(94° C. for 01:00, 52° C. for 01:00, 72° C. for 01:00);
1 cycle at 72° C. for 20:00 minutes.

The PCR conditions for the specific common detection of E. coli, B. subtilis and S. epidermidis are as follows:
1 cycle at 95° C. for 05:00 minutes;
35 cycles

(94° C. for 01:00, 62° C./60° C./64° C. respectively for 00:40, 72° C. for 01:10);
1 cycle at 72° C. for 20:00 minutes.

The PCR conditions for the specific nested PCR detection:
1 cycle at 95° C. for 05:00 minutes;
35 cycles

(94° C. for 01:00, 60° C. for 00:40, 72° C. for 01:10);
1 cycle at 72° C. for 20:00 minutes.

The PCR products are analyzed on agarose gel by comparison with a known scale DNA ladder (Gel Pilot 1 Kb or 100 by Plus Ladder, Qiagen, Ref. 239045).

### Results

### Influence of the Lysis Compositions on the Quantity and the Quality of the DNA Purified on a Membrane

A first approach consisted of using commercial kits such as those from Qiagen® or Dynal® for example. However, their use did not permit a sufficient quantity of nucleic acids to be purified from the microorganisms retained at the surface of the filtration membrane to enable amplification by PCR detectable on agarose gel (the number of cells contained in a micro-colony detectable by the Milliflex Rapid system corresponds to 100-1000 bacterial cells.)

A second approach consisted of using a lysis composition having as its formulation:
guanidine thiocyanate 4M,
Tris 50 mM
EDTA 25 mM

This composition was tested on different strains of microorganisms, then the genomic DNA purified according to the two methods:
precipitation according to the Modified Crude Susan method; or
by filtration on a cellulose membrane.

The results obtained according to the Modified Crude Susan method turned out to be disappointing due to the low reproducibility of the results: the PCR amplifications were of a variable intensity from one preparation to another, probably due to a loss of genetic material during the precipitation step.

As regards purification on a Microcon® membrane, this was a failure because the lysis composition perforated the cellulose membrane, due to a guanidium thiocyanate concentration which was apparently too high (4M).

On account of the results obtained, guanidium thiocyanate was not reused thereafter.

A third approach consisted of preparing and testing a series of compositions comprising guanidium chloride (GHCl). Detergents have been added to some of these compositions (Tween, Triton, NLS) and EDTA. These compositions have twice been independently tested on cells of the bacterium E. coli (gram-negative). The extractions of genomic DNA were carried out starting either from fresh cells originating from a diluted culture, or fresh cells originating from micro-colonies after filtration on a membrane, or micro-colonies filtered on a membrane then treated by bioluminescence according to the Milliflex® method, i.e. dead cells. In all cases, the genomic DNA content in the cell lysates was purified on a Microcon® cellulose membrane, then amplified by PCR, using so-called universal primers.

| Composition 1 | |
|---|---|
| Guanidium chloride | 6M |
| Tris | 10 mM |
| EDTA | 1 mM |
| Composition 2 | |
| Guanidium chloride | 6M |
| Tris | 10 mM |
| EDTA | 1 mM |
| Triton X100 | 1% final |
| Composition 3 | |
| Guanidium chloride | 6M |
| Tris | 10 mM |
| EDTA | 1 mM |
| Tween 20 | 1% final |
| Composition 4 | |
| Guanidium chloride | 6M |
| Tris | 10 mM |
| EDTA | 1 mM |
| N-lauroyl-sarcosine (NLS) | 1% final |
| Composition 5 | |
| Guanidium chloride | 6M |
| Tris | 10 mM |
| EDTA | 1 mM |
| N-lauroyl-sarcosine (NLS) | 0.5% final |
| Composition 6 | |
| Guanidium chloride | 6M |
| Tris | 10 mM |
| EDTA | 0.5 mM |
| N-lauroyl-sarcosine (NLS) | 1% final |
| Composition 7 | |
| Guanidium chloride | 6M |
| Tris | 10 mM |
| EDTA | 0.5 mM |
| N-lauroyl-sarcosine (NLS) | 0.5% final |

The amplification results are shown in the table in FIG. 1. The controls used for testing the inhibition effect of the lysis compositions on the PCR (PCR inhibition control) originate from samples into which pure bacterial DNA was introduced before purification on a membrane. These results indicate that only composition 7 made it possible to obtain satisfactory and reproducible results as regards the cells filtered on a membrane. This is verified for a number of cells comprised between 10 and 100 previously treated by bioluminescence or not, i.e. living or dead at the time of cell lysis. These experiments show the significant influence of the lysis composition on the effectiveness of the DNA purification on a membrane and its amplification by PCR.

### Universality, Specificity and Sensitivity of Detection

The protocol for the extraction and detection of genomic DNA developed using lysis composition No. 7 described above was applied to different microorganisms: Moulds, Yeasts, gram+ and gram− Bacteria, in order to verify the specificity and universality of the method according to the invention.

Universality

The detection results produced are presented in FIGS. 2 and 3A and 3B.

These results show that a detection of all the microorganisms tested can be obtained by use of the universal PCR primers, with a comparable level of signal amplification.

The nucleic acids of the bacteria Propionibacterium acnes ATCC 6919 (between 5 and 6 CFU incubated on TSA medium anaerobically at 32.5° C. for 39 hours) were extracted according to the same method. The DNA contained in this preparation of nucleic acids was amplified by PCR in the same manner as stated above, using the oligonucleotides Bact-F and Bact-R, which allow a universal detection of the bacteria. The results of the amplifications for the three nucleic acid preparations tested are shown in FIG. 7.

Specificity

PCRs using the specific internal primers of the following microorganisms: B. subtilis ATCC 6633, S. epidermidis ATCC 14990, and E. coli ATCC 25922 were carried out to verify the specificity of the detection, in particular when several types of cells are in a mixture.

A population of cells comprising these three bacterial species was thus prepared, then filtered on a membrane of the Milliflex Rapid® system, enabling their detection by ATP bioluminescence. The results of detection by ATP-bioluminescence for 3 different cell samples are shown in FIG. 4A. Then, the cells were treated with a lysis composition according to the invention (composition N°7) and their genomic DNA purified on a cellulose membrane as described previously. The results obtained by PCR with the universal primers and each pair of specific primers are shown in FIG. 4B.

In another experiment, two cell cultures, one of B. subtilis ATCC 6633 and the other of S. epidermidis ATCC 14990 were treated according to the Milliflex Rapid® detection protocol described previously (FIG. 5A results). The DNA of the bacteria was then extracted and purified on a membrane by using the lysis composition N°7 described previously, then amplified by PCR using the universal primers (FIG. 5B results).

The amplification products were then subjected to micro-sequencing. The results of the micro-sequencing (FIG. 5C) show that 100% of the amplification products correspond to the microorganism in culture.

Detection Sensitivity

In order to determine the detection threshold of the method according to the invention, 1 CFU of the bacterium E. coli and the yeast S. cerevisiae were placed on separate membranes. The membranes were enumerated by ATP bioluminescence using the Milliflex Rapid system, then the Genomic DNA was extracted and amplified, as indicated previously.

The results given in FIG. 6 show that a number of cells as low as that represented by 1 CFU can be amplified by PCR following the DNA extraction method according to the invention. This sensitivity is achieved both for the bacteria and for the yeast.

### Detection of the RNA Contained in the Nucleic Acid Preparations

HPA method (Hybridization Protection Assay)

The RNA contained in a nucleic acid preparation prepared starting from 1.6.10⁸ CFU of Propionibacterium acnes according to the method stated previously, were detected by bioluminescence following the HPA method.

This method is implemented using the MTC-NI RAPID DETECTION SYSTEM kit from Gen-Probe® (104574 Rev.C). This kit makes it possible to quantify the concentration of the ribosomal RNA of P. acnes available after cell lysis. The presence of RNA allows a single-strand DNA, comprising molecules of acridine ester, to be protected during the hybridization phase. Peroxide ions are then added and the reading is taken. The peroxide ions react with the acridine ester molecules, causing photon emission. The quantity of photons emitted is proportional to the quantity of RNA originally added. The light emitted is measured using a luminometer and the results are given in relative light units.

Table 4 compares the results obtained for the preparation extracted using the lysis composition No. 7 according to the invention, the preparation obtained using the lysis composition supplied with the MTC-NI kit, and for the negative and positive controls supplied in the MTC-NI kit.

TABLE 4
Results of RNA detection by HPA luminescence

| Sample | Relative light unit |
|---|---|
| Negative control | 769 |
| (Hybridization buffer only) | |
| Positive control | 65.097 |
| internal control of the kit) | |
| Lysis of P. acnes with | 9.923 |
| the composition of the MTC-NI kit | |
| Lysis of P. acnes with the composition | 33.438 |
| [Guanidium HCl 6M, Tris 5 mM, EDTA | |
| 0.5 mM, NLS 0.5%] | |

These results show that the preparation of nucleic acids obtained using the lysis composition according to the invention makes it possible to obtain a better detection than that obtained using the lysis composition supplied in the MTC-NI kit.

TMA Amplification Method (Transcription Mediated Amplification):

The RNA contained in nucleic acid preparations prepared from 30 and 300 CFU of Propionibacterium acnes according to the method indicated previously, were amplified and detected by the TMA technique described by Craig S Hill et al. [Molecular diagnostic testing for infectious diseases using TMA technology (2001) Expert Rev Mol Diagn. 1(4):445-55]. This technique consists, in a first step, of retro-transcribing the RNA molecules present in the sample, then in a second step, synthesizing the RNA fragments (isothermal amplification step) by transcription from the DNA produced in the first step.

The TMA technique was implemented using the reagents from the Milliprobe Pseudomonas aeruginosa detection kit (Millipore ref. GPPAE0100).

FIG. 8 represents the real-time detection of the amplification products synthesized in the reaction medium for each of the nucleic acid preparations according to the invention (30 and 300 CFU).

The preparation of nucleic acids corresponding to 30 CFU allows a satisfactory amplification almost equivalent to that corresponding to 300 CFU, demonstrating a very high level of effectiveness for extraction of the RNA of P. aeruginosa by the lysis composition No. 7.

1. Method for the detection of one or more cells in a liquid or gaseous medium, comprising the following steps:
a) filtering a sample of the liquid or gaseous medium through a membrane to retain the cells contained in said sample on said membrane;
b) treating the cells retained in step a) with guanidium chloride and N-Lauroyl-sarcosine (NLS) in solution, in order to obtain a cell lysate containing the nucleic acids released from said cells;
c) separating the nucleic acids are from the cell lysate obtained in step b) by filtration of said lysate through said membrane, or through another membrane;
d) recovering the nucleic acids retained by the membrane in step c) in water or in a weakly ionized aqueous solution;
e) detecting the nucleic acids recovered in step d).

2. Method according to claim 1, wherein said detection of the nucleic acids in step e) is carried out by specific hybridization of all or part of the nucleic acids recovered in step d) using a probe or a specific marked primer.

3. Method according to claim 1, wherein said detection of the nucleic acids in step e) comprises a step of amplification of all or part of the nucleic acids recovered in step d).

4. Method according to claim 3, wherein said amplification step comprises a polymerisation chain reaction (PCR).

5. Method according to claim 4, wherein said amplification step comprises an isothermal amplification.

6. Method according to claim 1, wherein said detection of the nucleic acids in step e) comprises a step of retrotranscription of the RNA to DNA.

7. Method according to claim 1, wherein the membrane used to separate the nucleic acids of the lysis composition in step c) is different from the one used in step a).

8. Method according to claim 1, wherein said NLS is used in solution at a concentration between 0.1 and 1%.

9. Method according to claim 1, wherein said membrane used in step a) is placed in contact with a nutrient medium so that the microorganisms can grow on the surface of said membrane between steps a) and b).

10. Method according to claim 1, wherein a detection step, making it possible to count the microorganisms, is carried out between steps a) and b), by reacting the ATP extracted from the microorganisms with a bioluminescent reactant.
